(19) United States
(12) Patent Application Publication
(10) Pub. No.: US 2022/0215957 A1
(43) Pub. Date: Jul. 7, 2022

(54) Digital Nurse for Symptom and Risk Assessment

(71) Applicant: Liyfe, Inc., Woodbridge, CT (US)

(72) Inventors: Xueyan Chen, Woodbridge, CT (US); Xiaoli Tang, Woodbridge, CT (US)

(21) Appl. No.: 17/567,778

(22) Filed: Jan. 3, 2022

Related U.S. Application Data
(60) Provisional application No. 63/134,060, filed on Jan. 5, 2021.

(51) Int. Cl. G16H 50/20; G16H 10/20; G06N 20/20; H04L 51/02

(52) U.S. Cl. CPC G16H 50/20; H04L 51/02; G06N 20/20; G16H 10/20

(57) ABSTRACT

A system and method for intelligent symptom assessment through a machine learning-driven digital assistance platform are disclosed. The system is configured to process a user input received from a user device communicating with a chatbot environment, the user input indicating a user request for assessing a symptom in the chatbot environment, generate one or more questions related to the symptom, and communicate the one or more questions to the user device in the chatbot environment, receive, in the chatbot environment, responses to the one or more questions provided by the user, collect additional medical information associated with the user, and determine one or more parent symptoms or complications associated with the symptom based on the one or more responses provided by the user and the additional medical information of the user.

### CROSS REFERENCE TO RELATED APPLICATION

The application claims priority of U.S. provisional application No. 63/134,060 filed on Jan. 5, 2021, and entitled “Patient-Reported Outcome Platform for Symptom Management.” This application is hereby incorporated by reference in its entirety.

### TECHNICAL FIELD

The present disclosure is directed to systems and methods for digital medical assistance, and in particular to systems and methods for intelligent patient symptom assessment through a machine learning-driven digital assistance platform.

### BACKGROUND

Most cancer patients experience side effects throughout the entire treatment course, which are also called symptoms. These cancer symptoms are either treatment-related or disease-related. Managing these symptoms with existing resources to keep patients out of hospitals has always been a challenge. In fact, 53% of emergency room (ER) visits are unnecessary and can be avoided. By manually tracking symptoms at a certain frequency (e.g., once per week), it can increase the survival probability and quality of life of patients, and reduce ER visits and hospitalization. For example, patients can visit outpatient facilities at a certain frequency, to allow nurses or other healthcare providers to manually follow a standard triage protocol to ask patients a set of questions to obtain information necessary to triage the patients, e.g., to provide clinical advice, send the patients to see an oncologist, send patients to ER, etc. However, this triage process is mostly repeatable work, labor-intensive, and time-consuming. Hospitals need to have a dedicated triage team to handle the load, which often involves additional cost. In addition to cost, there is also national nurse shortage.

Patient-reported outcome (PRO) platforms have been recently developed, which allow patients to report and manage symptoms, and communicate with and receive care from a clinical team. These PRO platforms empower not only patients, but also clinicians, which makes symptom management more efficient. Using these PRO platforms, patients can report symptoms without frequent visits to outpatient facilities. For example, in a scenario that a patient gets up vomiting at 2 am, the patient may directly report the symptom through a PRO platform, which is convenient and time-saving. However, the current PRO platforms have certain limitations. Since it requires a nurse to manually perform symptom assessment in order for the oncologists to treat patients, it requires either patients to travel to the hospital or call the nurses. It can easily take 1-2 hours to assess one PRO. Due to the cost and national nurse shortage, it is impossible for hospitals to handle reported PRO. As a result, PRO is not widely implemented in the clinic.

### SUMMARY

To address the aforementioned shortcomings, a method and a system for intelligent symptom assessment through a machine learning-driven digital assistance platform are provided.

In one aspect, a system for intelligent symptom assessment through a machine learning-driven digital assistance platform includes a processor, and a memory, coupled to the processor, configured to store executable instructions. The instructions, when executed by the processor, cause the processor to process a user input received from a user device communicating with a chatbot environment, the user input indicating a user request for assessing a symptom in the chatbot environment, generate one or more questions related to the symptom and communicate the one or more questions to the user device in the chatbot environment, receive, in the chatbot environment, responses to the one or more questions provided by the user, collect additional medical information associated with the user, and determine one or more parent symptoms or complications associated with the symptom based on the one or more responses provided by the user and the additional medical information of the user.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The drawing figures depict one or more embodiments in accordance with the present teachings, by way of example only, not by way of limitation. In the figures, like reference numerals refer to the same or similar elements. Furthermore, it should be understood that the drawings are not necessarily to scale.

FIG. 1 illustrates a block diagram of an example digital nursing system, according to embodiments of the disclosure.

FIG. 2 illustrates a block diagram of an example computing device included in a digital nursing system, according to embodiments of the disclosure.

FIG. 3 illustrates a block diagram of example components for a digital nursing application included in a digital nursing system, according to embodiments of the disclosure.

FIG. 4 illustrates a high-level conceptual framework of a prediction model to predict parent symptoms and complications, according to embodiments of the disclosure.

FIG. 5 illustrates an example workflow for prediction of parent symptoms and/or complications associated with a reported symptom, according to embodiments of the disclosure.

FIGS. 6A-6B collaboratively illustrate an example method for assessing a symptom reported by a patient, according to embodiments of the disclosure.

FIGS. 7A-7O illustrate example patient-side user interfaces under different scenarios, according to embodiments of the disclosure.

FIGS. 8A-8C illustrate example healthcare provider-side user interfaces, according to embodiments of the disclosure.

### DETAILED DESCRIPTION

In the following detailed description, numerous specific details are set forth by way of examples in order to provide a thorough understanding of the relevant teachings. However, it should be noted that the present teachings may be practiced without such details. In other instances, well-known methods, procedures, components, and/or circuitry have been described at a relatively high level, without detail, in order to avoid unnecessarily obscuring aspects of the present teachings.

The present disclosure provides technical solutions to address the technical problems in current PRO platforms. The technical solutions provide a digital nursing system that can automatically assess a symptom reported by a patient. In addition, the digital nursing system disclosed herein may allow a patient to report a symptom under a clear guide, which may prevent irrelevant information from being collected or important information being missed during a symptom report. Further, the technical solutions disclosed herein may also enable certain actions/plans to be timely taken for a reported severe or emergent symptom. For example, based on the severity determined during the symptom assessment, the disclosed digital nursing system may send an immediate alert to one or more corresponding parties in charge, so that timely care can be provided to a patient if the patient is found to be in a very severe or emergent medical condition.

The disclosed digital nursing system shows certain technical improvements when compared to other existing PRO platforms. First, the disclosed digital nursing system may provide an instant and automatic assessment for a reported symptom, which then does not require a nurse to manually perform symptom and risk assessment. Second, the disclosed digital nursing system may adaptively provide questions during a symptom assessment process, driven by the machine learning-based models. This then prevents a patient from submitting unrelated information or submitting multiple reports due to certain important information being missed in the initial report(s), which then saves the computing resources including bandwidths allocated for online healthcare management. Third, the disclosed digital nursing system may provide improved user interfaces for a symptom reporting process by including intelligently prepared answers to questions included in a chatbot user interface, which then prevents a patient from repeatedly typing answers to the questions or making certain corrections to undesirable answers provided by the system. This benefit becomes more obvious especially when a cell phone, smart watch, or another small mobile device is being used for a symptom assessment process, where these devices are known to be not convenient for frequent typing. The technical solutions disclosed herein, therefore, show an improvement in the functioning of computing devices, particularly those configured for online management of patient symptoms.

The benefits and advantages described herein are not all-inclusive and many additional features and advantages will be apparent to one of ordinary skill in the art in view of the figures and the following descriptions.

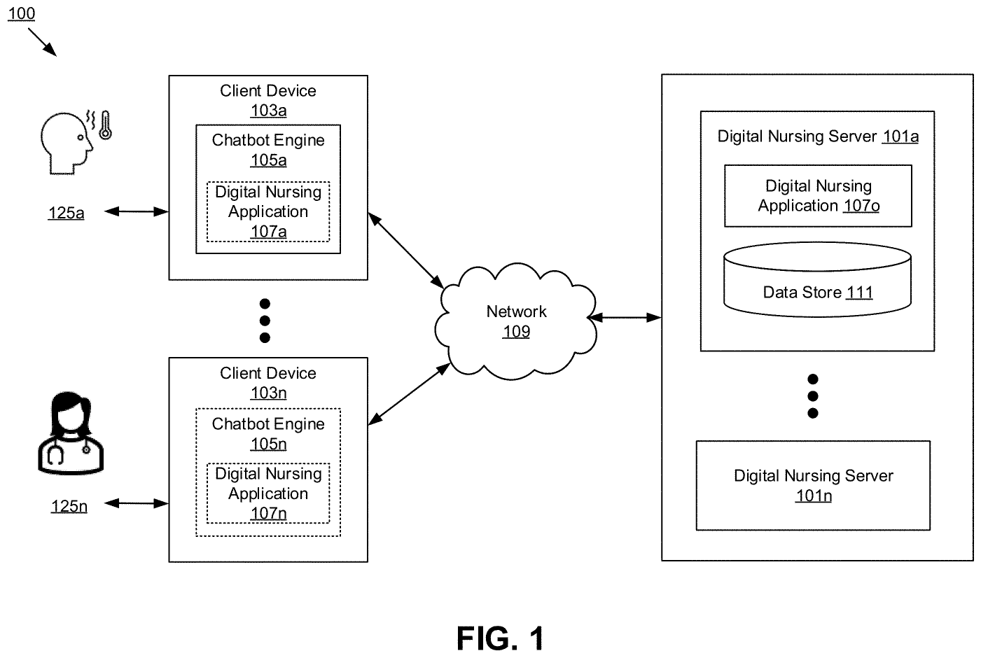

FIG. 1 illustrates a block diagram of an example digital nursing system 100, according to embodiments of the disclosure. In implementations, a digital nursing system 100 may take the form of hardware and/or software components running on hardware. In some embodiments, a digital nursing system 100 may provide an environment for software components to execute, evaluate operational constraint sets, and utilize resources or facilities of the digital nursing system 100. For instance, software (e.g., applications or apps, operational instructions, modules, etc.) may be running on a processing device, such as a computer, mobile device (e.g., smartphone/phone, smartwatch, fitness tracker, tablet, laptop, personal digital assistant (PDA), patient monitoring device, etc.) and/or any other electronic device. In other instances, the components of a digital nursing system 100 disclosed herein may be distributed across and executable by multiple devices. For example, an input may be entered on a client device, and information may be processed or accessed from other devices (e.g., servers or other client devices, etc.) in a network.

As illustrated, a digital nursing system 100 may include client devices 103a-103n (collectively or individually referred to as client device 103), distributed network 109, and a distributed server environment comprising one or more servers, such as digital nursing servers 101a-101n (collectively or individually referred to as digital nursing server 101). Each client device 103 may be associated with a user 125a or 125n (collectively or individually referred to as user, individual, client, or patient 125). One of the skilled in the art will appreciate that the scale of digital nursing system 100 may vary and may include additional or fewer components than those described in FIG. 1. In some embodiments, interfacing between components of a digital nursing system 100 may occur remotely, for example, where components of the digital nursing system 100 may be distributed across one or more devices of a distributed network.

Client devices 103a-103n may be configured to receive input via a user interface component or other input means. Examples of input may include voice, visual, touch, or text input, etc. In some embodiments, one or more portions of the input may correspond to symptoms associated with a user 125 (e.g., a fever associated with a user 125a). Client devices 103a-103n may store the symptom data and/or provide access to data sources comprising the symptom data or other medical information of a patient for one or more people/entities/devices. The data sources may be located on, or accessible to, digital nursing servers 101a-101n via a network 109. As an example, such data may be locally stored on the client devices 103a-103n, or on one or more of the digital nursing servers 101a-101n, e.g., in the data store 111 coupled to a digital nursing server 101.

In some embodiments, user input including the symptom data may be received through a chatbot provided on a client device 103. Accordingly, a client device 103 may include a chatbot engine 105a or 105n (collectively or individually referred to as chatbot engine 105 or simply chatbot 105) configured to enable chat communication. A chatbot engine 105 may be a machine learning-based conversational dialog engine built in Python or another computing language that makes it possible to generate responses/questions based on collections of known conversations. As a software agent that can perform tasks or services for an individual, a chatbot engine 105 may interact directly with a user 125 to receive input from the user (e.g., commands, in the form of speech or text) and provide output to the user (e.g., communicate, in the form of speech or text). As discussed herein, various chatbots may be integrated with a digital nursing system 100, which would include a repository of task-specific bots for various consumer task completion scenarios. In one example, a chatbot engine 105 may include a digital nursing application 107a, 107n, or 107o (collectively or individually referred to as digital nursing application 107), which makes the chatbot engine 105 a specialized chatbot for simulating the way a nurse (or other healthcare providers) would behave as a conversational partner. In some embodiments, other types of chatbot engines, such as Apple's Siri® and Amazon's Alexa®, may be also included in a client device 103. In some embodiments, the specialized chatbot may be embodied as a cloud-based application available in iOS, Android, Windows App, or in a web version, etc. In some embodiments, a client device 103 may not have a chatbot engine 105. For instance, a client device 103n may be associated with a healthcare provider 125n, and the client device 103n may not have a chatbot engine 105n, but rather have a web version of a digital nursing system that contains user interfaces for monitoring patients, as described in detail later.

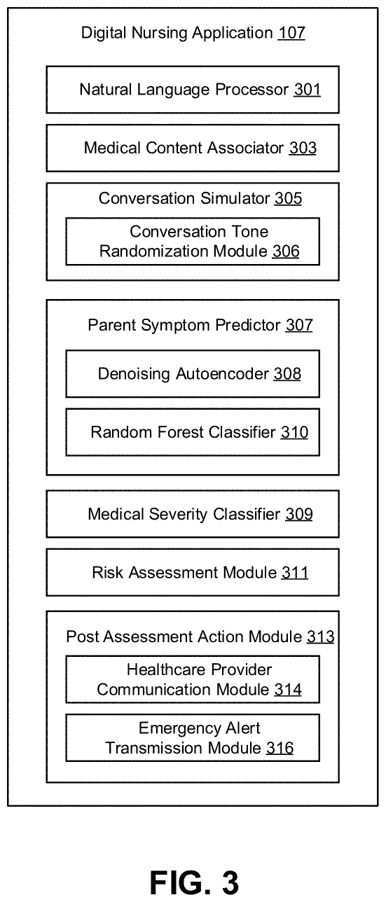

To configure a chatbot 105 specialized for digital nursing, a digital nursing application 107 may be configured to implement interaction rules specifically designed to deal with healthcare-related chat communications. These interaction rules may include rules for identifying a proper communication tool (e.g., text or voice chat) for a patient, rules for identifying specific questions to ask following a symptom report, rules for determining a pattern (e.g., a survey or a plain text, a voice, etc.) to present these specific questions to a patient, and so on. For example, for each symptom reported by a patient, a digital nursing application 107 may determine possible diseases associated with the symptom, and determine what questions to ask for the reported symptom. In some embodiments, one or more machine learning models may be included in a digital nursing application 107 to identify the most proper questions to ask for a reported symptom. In some embodiments, besides setting specific interaction rules for chat communication, a digital nursing application 107 may implement other symptom assessment-related activities, such as identifying intention/context of the chat communication, determining a parent symptom and/or complications for a reported symptom based on the chat communication, determining a medical severity for a reported symptom, assessing the risk for a reported symptom, as further described more in details in FIG. 3. In some implementations, each instance of digital nursing applications 107a . . . 107o includes one or more components as depicted in FIG. 3, and may be configured to fully or partially perform the functionalities described therein depending on where the instance resides. For instance, a special instance of the digital nursing application may be included in a digital nursing server and responsible for handing chat communications between patients and the digital nursing system 100, while another instance of the digital nursing application may be included in the same or different digital nursing server and responsible for handing healthcare provider activities in healthcare management included in the digital nursing system 100. In some embodiments, a digital nursing application 107 may be not necessarily included in a chatbot engine 105 on a client device, but can be a standalone application that renders a web version of a digital nursing system on a client device. For instance, a client device 103n associated with a healthcare provider 125n may have a web version digital nursing application not included in a chatbot 105.

A digital nursing server 101 may be a cloud server that possesses larger computing capabilities and computing resources than a client device 103, and therefore may perform more complex computation than the client device 103 can. For example, an instance of digital nursing application 107o included in a server 101 may perform a complicated decision process to determine parent symptoms and associated complications for a reported symptom and severity for each symptom and complication. For another example, a digital nursing application 107 included in a client device 103 may perform a simple decision process to determine which entity to contact if a reported symptom is severe. The different instances of digital nursing applications may communicate with other components of the digital nursing system 100 via a network 109.

Network 109 may be a conventional type, wired and/or wireless, and may have numerous different configurations, including a star configuration, token ring configuration, or other configurations. For instance, the network 109 may include one or more local area networks (LAN), wide area networks (WAN) (e.g., the Internet), public networks, private networks, virtual networks, mesh networks, peer-to-peer networks, and/or other interconnected data paths across which multiple devices may communicate. The network 109 may also be coupled to or include portions of a telecommunications network for sending data in a variety of different communication protocols. In some implementations, the network 109 includes Bluetooth® communication networks or a cellular communications network for sending and receiving data including via short messaging service (SMS), multimedia messaging service (MMS), hypertext transfer protocol (HTTP), direct data connection, wireless application protocol (WAP), email, etc.

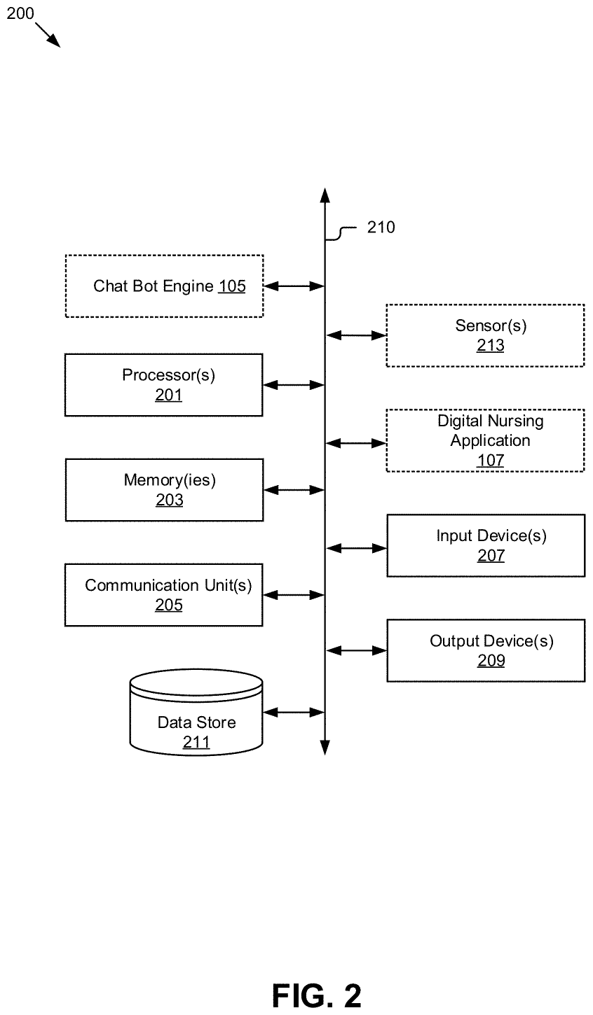

FIG. 2 illustrates a block diagram of an example computing device 200 included in a digital nursing system 100, according to embodiments of the disclosure. The example computing device 200 may represent the architecture of a digital nursing server 101 or a client device 103. As illustrated, a computing device 200 may include one or more processors 201, one or more memories 203, one or more communication units 205, one or more input devices 207, one or more output devices 209, and a data store 211. In some embodiments, a computing device 200 may further include a chatbot engine 105, a digital nursing application 107 coupled to the chatbot engine 105. In embodiments where a computing device 200 serves as a client device 103, one or more sensors 103 (e.g., imaging or voice-related sensors, healthcare monitoring related sensors) may be also included in the computing device 200. In some embodiments, different components of a computing device 200 are communicatively coupled by a bus 210.

Processor(s) 201 may execute software instructions by performing various input/output, logical, and/or mathematical operations. Processor(s) 201 may have various computing architectures to process data signals, including for example a complex instruction set computer (CISC) architecture, a reduced instruction set computer (RISC) architecture, and/or an architecture implementing a combination of instruction sets. Processor(s) 201 may be physical and/or virtual, and may include a single core or plurality of processing units and/or cores. In some embodiments, processor(s) 201 may be capable of generating and providing electronic display signals to a display device (not shown), supporting chatbot communications, capturing and analyzing images, capturing and converting voice, performing complex tasks including various types of feature extraction and classification, etc. In some embodiments, processor(s) 201 may be coupled to the memory(ies) 203 via bus 210 to access data and instructions therefrom and store data therein. Bus 210 may couple processor(s) 201 to other components of computing device 200 including, for example, memory(ies) 203, communication unit(s) 205, sensor(s) 213, chatbot engine 105, digital nursing application 107, input device(s) 207, output device(s) 209, and/or data store 211.

Memory(ies) 203 may store and provide access to data to other components of a computing device 200. In some embodiments, memory(ies) 203 may store instructions and/or data that may be executed by the processor(s) 201. For example, depending on the configuration of the computing device 200, memory(ies) 203 may store one or more instances of a chatbot engine 105 and/or a digital nursing application 107. Memory(ies) 203 are also capable of storing other instructions and data, including, for example, an operating system, hardware drivers, other software applications, user profiles of patients, symptoms reported by patients, interaction rules for chatbot engines, etc.

Memory(ies) 203 may include one or more transitory or non-transitory computer-usable (e.g., readable, writeable, etc.) media, which may be any non-transitory apparatus or device that may contain, store, communicate, propagate or transport instructions, data, computer programs, software, code, routines, etc., for processing by or in connection with the processor(s) 201. For example, memory(ies) 203 may include, but are not limited to, one or more of a dynamic random access memory (DRAM) device, a static random access memory (SRAM) device, a discrete memory device (e.g., a PROM, FPROM, ROM), a hard disk drive, an optical disk drive (CD, DVD, Blue-ray™, etc.). It should be understood that memory(ies) 203 may be a single device or may include multiple types of devices and configurations distributed locally or remotely (e.g., cloud storage).

Communication unit(s) 205 may be configured to transmit data to and receive data from other computing devices to which they are communicatively coupled using wireless and/or wired connections (e.g., via the network 109). Communication unit(s) 205 may include one or more wired interfaces and/or wireless transceivers for sending and receiving data. Communication unit(s) 205 may couple to the network 109 and communicate with other computing nodes, such as the client device(s) 103, and/or digital nursing server(s) 101, etc. The communication unit(s) 205 may exchange data with other computing nodes using standard communication methods.

Bus 210 may include a communication bus for transferring data between components of a computing system 200 or between computing systems, a network bus system including network 109 and/or portions thereof, a processor mesh, a combination thereof, etc. In some embodiments, bus 210 may represent one or more buses including an industry-standard architecture (ISA) bus, a peripheral component interconnect (PCI) bus, a universal serial bus (USB), or some other buses known to provide similar functionality. Additionally and/or alternatively, the various components of computing device 200 may cooperate and communicate via a software communication mechanism implemented in association with the bus 210. The software communication mechanism may include and/or facilitate, for example, inter-process communication, local function or procedure calls, remote procedure calls, an object broker (e.g., common object request broker architecture (CORBA)), direct socket communication (e.g., TCP/IP sockets) among software modules, user datagram protocol (UDP) broadcasts and receipts, HTTP connections, etc. Further, any or all of the communication could be secure (e.g., SSH, HTTPS, etc.).

Data store(s) 211 may be included in the one or more memories 203 of the computing device 200 or in another computing device and/or storage system distinct from but coupled to or accessible by the computing device 200. In some embodiments, the data store(s) 211 may store data in association with a database management system (DBMS) operable by the servers 101 and/or the client devices 103. For example, the DBMS could include a structured query language (SQL) DBMS, a NoSQL DMBS, etc. In some instances, the DBMS may store data in multi-dimensional tables comprised of rows and columns, and manipulate, e.g., insert, query, update and/or delete, rows of data using programmatic operations.

Input device(s) 207 may include any standard devices configured to receive a variety of control inputs (e.g., gestures, voice controls) from a user 125 or other devices. Non-limiting example input device 207 may include a touch screen (e.g., LED-based display) for inputting texting information, making a selection, and interacting with the user 125; motion-detecting input devices; audio input devices; other touch-based input devices; keyboards; pointer devices; indicators; and/or any other inputting components for facilitating communication and/or interaction with the user 125 or the other devices. For example, the input device(s) 207 may include a touch-screen, microphone, a front-facing camera, a rear-facing camera, and/or motion sensors, etc. The input device(s) 207 may be coupled to the computing device 200 either directly or through intervening controllers to relay inputs/signals received from users 125 and/or sensor(s) 213.

Output device(s) 209 may include any standard devices configured to output or display information to a user 125 or other devices. Non-limiting example output device(s) 209 may include a touch screen (e.g., LED-based display) for displaying a chatbot to the user 125, an audio reproduction device (e.g., speaker) for delivering sound information to the user 125, a display/monitor for presenting texting or graphical information to the user 125, etc. The outputting information may be text, graphic, tactile, audio, video, and other information that may be understood by the user 125 or the other devices, or may be data, logic, programming that can be readable by the operating system of the computing device 200. The output device(s) 209 may be coupled to the computing device 200 either directly or through intervening controllers.

Sensor(s) 213 may include any type of sensors suitable for a client device 103. The sensor(s) 213 may be configured to collect any type of data suitable to determine symptoms and other health conditions of patients. Non-limiting examples of the sensor(s) 103 include various optical sensors (CCD, CMOS, 2D, 3D, light detection and ranging (LIDAR), cameras, etc.), audio sensors, motion detection sensors, barometers, altimeters, thermocouples, heart rate sensors, pulse sensors, moisture sensors, IR sensors, radar sensors, other photo sensors, gyroscopes, accelerometers, speedometers, geo-location sensors, transceivers, sonar sensors, ultrasonic sensors, touch sensors, proximity sensors, etc.

The chatbot engine 105 and the coupled digital nursing application 107 may be included if the computing device 200 serves as a client device (e.g., a patient device) 103. The functions of the chatbot engine 105 and the coupled digital nursing application 107 have been briefly described in FIG. 1, and will be described more in details below in FIG. 3.

FIG. 3 illustrates a block diagram of example components for a digital nursing application included in a digital nursing system, according to embodiments of the disclosure. As illustrated, a digital nursing application 107 may include a natural language processor 301, a medical content associator 303, a conversation simulator 305, a parent symptom predictor 307, a medical severity classifier 309, and a risk assessment module 311, and a post assessment action module 313. In some embodiments, the conversation simulator 305 may additionally include a conversation tone randomization module 306, the parent symptom predictor 307 may additionally include a denoising autoencoder 308 and a random forest classifier 310, and the post assessment action module 313 may additionally include a healthcare provider communication module 314 and an emergency alert transmission module 316.

Natural language processor 301 may be configured to parse user input (e.g., text, image, or voice input) to predict or identify user intent, according to embodiments of the disclosure. In some embodiments, when a patient reports a symptom through a chatbot 105, a symptom may be able to be selected from a list of available symptoms presented to the patient, and thus the patient just selects a symptom from the list for reporting. In some embodiments, a to-be-reported symptom may be not found from the list, and thus the patient may require to report the symptom through a text, image, voice, or other types of input. Natural language processor 301 included in the digital nursing application 107 may be configured to identify user intent, including identifying a to-be-reported symptom from the user input. To achieve such functions, natural language processor 301 may include certain text and speech processing components or modules (not shown), where each component or module may be responsible for one type of input processing. For instance, the natural language processor 301 may include an optical character recognition module configured to determine corresponding text from an image input by a patient (e.g., a handwritten symptom), a speech recognition module configured to determine the textual representation of a voice input (e.g., an orally reported symptom), an image recognition module configured to determine a symptom from an input image (e.g., a vomiting image or a bleeding image) through object recognition or scene reconstruction. Other possible modules or components included in a natural language processor 301 may include certain syntactic analysis modules and/or lexical semantics modules for content parsing, sentence breaking, keyword identification, etc. These different modules or components collaboratively allow a prediction of the user intent (e.g., identify a to-be-reported symptom) based on various types of input received from a patient.

Medical content associator 303 may be configured to determine suitable medical content associated with a reported symptom. For instance, based on the identified symptom reported by a patient, the medical content associator 303 may identify a list of diseases associated with the symptom, and even more specifically in which stage the symptom may occur in a disease. In some embodiments, the medical content associator 303 may further retrieve the user profile and medical information (e.g., medical history) of the patient in identifying a specific disease for the symptom reported by the patient. For instance, the reported symptom may match a disease previously diagnosed for the patient based on the medical record of the patient. In some embodiments, based on the identified disease, the medical content associator 303 may determine what supplemental information is necessary to assess the severity of the reported symptom. For example, if vomiting is reported, the supplemental information may include how long the vomiting lasts, how many times the vomiting occurred, there is any pain, and where is the pain if there is any, etc. These questions may be then presented to the patient through chat communication, so as to collect the necessary supplemental information.

In some embodiments, the medical content associator 303 may develop questions according to certain standards in healthcare practice, such as National Cancer Institute (NCI)'s patient-reported outcome (PRO)-common terminology criteria for adverse events (CTCAE) standard and oncology nurse triage protocols. Accordingly, two sets of questions may be developed by the medical content associator 303 according to some embodiments: onset question set and symptom assessment question set. The onset question set may include a set of questions that discover the date and time when a patient initially experienced a reported symptom, whether the symptom happened gradually or suddenly, etc. The symptom assessment questions may provide certain parameters for determining the severity of the reported symptom. For instance, the symptom assessment questions may include how severe a patient is, how painful the patient feels about the symptom, how a symptom affects the patient's daily activities, etc.

In some embodiments, the assessment questions for the same symptom from the same cancer type but different patients may be different. For instance, if a patient reported leg swelling, it is important to ask if the swelling is symmetrical or not. Depends on the patient's answer, the next generated question would be different. If a patient reported lack of appetite, the assessment will include eating and drinking situations, and if the patient loses weight, the assessment will include what the weight is, etc. Similarly, different patients' answers will lead to a different question to be asked next. Here the following are some example questions that may be used by the medical content associator 303 in developing a symptom assessment set:

Normality
What is the normal situation for the patient?
Is there any medical history of this symptom?

Region/Radiation
Where does the symptom occur?
What is the progressing pattern?

Quality
Details of the symptoms such as the feeling, physical pattern, smell, etc.

Provoking/Palliating
What could be the cause?
What makes it better or worse?

Grade
Grade the symptom. Depending on the symptoms, different standards are used. For instance, pain is graded using the PRO-CTCAE grading system.

Impact
How does it impact a patient's Activities of Daily Living (ADLs)? There are six basic ADLs: eating, bathing, getting dressed, toileting, mobility, and continence.

Associated symptoms
What other symptoms occur at the same time?

Alleviating factors
What medication or methods have the patient already tried to alleviate the symptom?

Additional information
Let the patient leave additional notes.

In some embodiments, for each question developed for the online chat communication, patients may be asked to provide their own answer. In some embodiments, however, patients may be provided with a set of answers to choose from. For instance, when assessing a drinking situation, the answer choices may be also provided for a question how often a patient drinks, as shown in the following:

I drink 8+ glasses of fluid per day.

I drink 3-8 glasses of fluid per day.

I drink 1-3 glasses of fluid per day.

I am not able to drink any fluid in the last 24 hours.

In response, the patient may simply select one of the provided answers, which may save the time and sources required to complete an online chat communication in the supplemental information collection, thereby releasing the resources for other busy online healthcare management.

It is to be noted that the above-described assessment questions are merely for exemplary purposes. For a specific reported symptom, the exact content of each question may vary, based on the reported symptom. In addition, for each patient, the exact content of each question may be also different. For example, for the same symptom severity, some patients may feel more severe, and some might feel less severe. Although it is important to understand how a patient feels, it is also important to have an objective assessment of the severity. To accommodate the patient distinction, the medical information of each patient may be retrieved in determining the exact content of each question for a specific patient, especially the content for a set of selectable answers to each question. In some embodiments, once the exact content of the patient-specific questions is determined, the assessment questions may be then presented to the patient during the symptom assessment.

Conversation simulator 305 may be configured to emulate human conversation with a user (e.g., the patient reporting the symptom), for example, communicate information such as the assessment questions for collecting the supplemental information in response to user input, or prompt the user for additional information. The assessment questions may be presented to the user one-by-one in plain text, in a survey format, by voice, by text, etc. When the questions are presented to the user, the user may respond to these questions through a chatbot. These responses may be then collected for insight analysis, so that the supplemental information for determining the severity and risk of the reported symptom, as well as the potential parent symptom and/or complications associated with the reported symptom, as further described later.

In some embodiments, the conversational simulator 305 may modify the assessment questions to be presented to a patient in real-time during the emulated chat communication. For instance, if a patient's responses to the first few questions indicate that the symptom may be not associated with a previously identified disease, but rather possibly point to a new disease not previously diagnosed for the patient, the conversion simulator 305 may adjust the questions to be more related to the new disease, and present adjusted questions to the patient. The conversation simulator 305 may achieve this by communicating with the medical content associator 303 to develop a new set of assessment questions. By real-time monitoring and evaluating each response from the user and dynamically adjusting assessment questions, it can be ensured that only relevant questions be asked during the symptom assessment, thereby saving the resources for online healthcare management. For instance, it may avoid another round of online chat communication (which is necessary if the first round of assessment questions are later found to be not sufficient by a healthcare provider).

In some embodiments, the conversational simulator 305 may further include a conversation tone randomization module 306 configured to randomize the conversational tone to make the online chat communication resemble a human conversation. For instance, after tone randomization, the conversation simulator 305 may present a question to be more casual, such as “I see. Are you experiencing . . . ?” “Umm . . . ” “Glad to hear that . . . ” “I hear you . . . ” In some embodiments, if voice communication is enabled and used for chat communication during symptom assessment (e.g., when a patient cannot read and/or input text), the tone randomization module 306 may even check the user profile of a patient and determine a local accent, which can be then incorporated into the chat communication, to allow the patient to better understand the chat content provided by the chatbot, thereby preventing important information from being missed during the assessment. In some embodiments, based on the determined severity and sentimental indication of the language or tone used in the chat commination, the tone randomization module 306 may even add certain sentimental language to the chat communication such as, “no worry, we will find out . . . ” to relax the patient. The objective of the conversation tone randomization module 306 is to make the whole symptom reporting process more accurate, smoother for the patient, and more comfortable and less stressful for the patient.

Continuing in FIG. 3, parent symptom predictor 307 may be configured to predict the parent symptom(s) and the complication(s) associated with a reported symptom based on the supplemental information collected through the chat communication along with the patient's medical history and background (e.g., diagnosis, medication, treatment, etc.).

To better explain the functions of the parent symptom predictor 307, the meaning of certain terms used in the specification is provided as follows:
A symptom is an observed or detectable sign such as pain, fatigue, or fever.
A reported symptom is a symptom reported by a patient. Typically, it is the one that bothers the patient the most.
Associated symptoms are the ones associated with a reported symptom. The associated symptoms typically happen along with the reported symptom.
A parent symptom is the cause of a reported symptom. It is possible that the reported symptom is the parent symptom. Often, however, the parent symptom is different from the reported symptom. For instance, fatigue can be caused by insomnia, headache, mal-nutrition, etc.
A complication is an unfavorable result of a disease or treatment such as anemia, intestinal obstruction, and pneumonitis. A complication often causes multiple symptoms.
Medical condition refers to a patient's overall condition, including the reported symptom, associated symptoms, parent symptoms, and potential complications.
   It is to be noted that symptom, complication, medical condition severities may be all described as non-urgent, urgent, and emergent. Non-urgent and urgent medical conditions typically do not need immediate care, but continual monitoring is needed. Care teams may need to see patients the next day or week after a symptom report. Emergent medical condition, as suggested by its name, does need immediate attention and/or action for the safety of a patient.

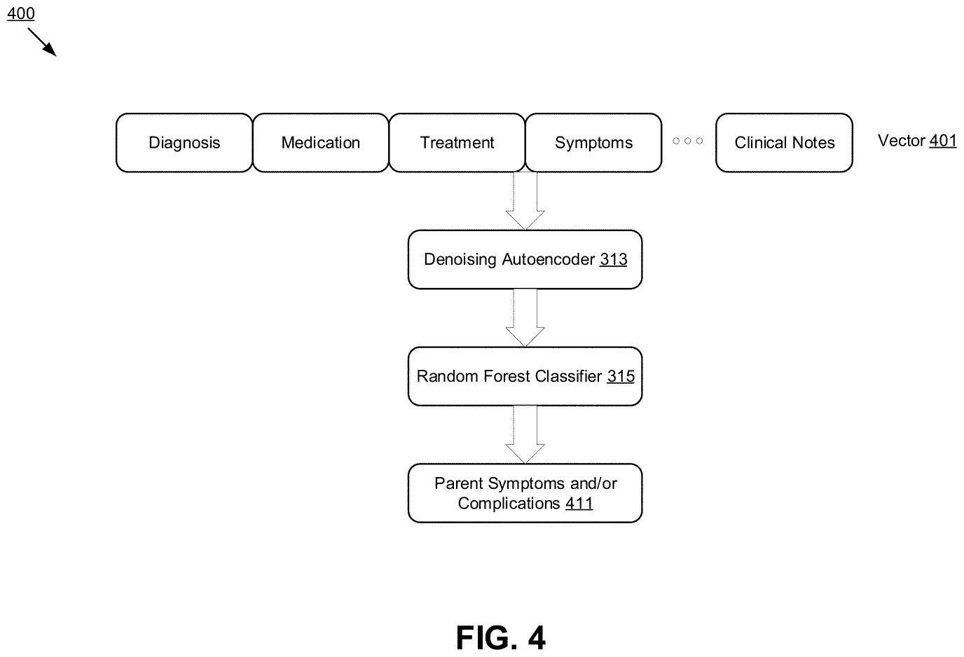

To identify the potential parent symptoms and/or complications associated with a reported symptom, the parent symptom predictor 307 may further include a prediction model developed based on a denoising autoencoder 308 and a random forest classifier 310, as illustrated in FIG. 3, and as further described in detail below in FIG. 4.

FIG. 4 illustrates a high-level conceptual framework 400 of a prediction model to predict parent symptoms and complications, according to embodiments of the disclosure. In the figure, a patient may be presented as a vector 401 containing information features such as diagnosis, medication, treatment, symptoms, demographic, symptom history, complication history, lab reports, and/or clinical notes, etc. These information features may be identified through the chat communication during the symptom report, or may be collected from the database related to patient information, including personal information and/or medical information. These information features may be first fed into the denoising autoencoder 308 for feature extraction.

The denoising autoencoder 308 may be a denoising autoencoder that is configured to reconstruct the input patent data from a noise version of the initial data 401 in order to prevent overfitting. Autoencoder is a type of neural network that can be used to learn a compressed representation of input data. To prevent overfitting, a three-layer denoising autoencoder may be applied, which itself may include a three-layer encoder and a three-layer decoder. The three-layer encoder may extract features from the input data 401 and the three-layer decoder may attempt to reconstruct the input from the extracted features. When training the prediction model, the algorithm searches for parameters that minimize the reconstruction error, that is, the difference between the reconstruction and input data. After training, the three-layer encoder model may be saved and used for feature extraction, and the three-layer decoder may be discarded.

It is to be noted that in real applications, patient data entries vary from person to person. In addition, there are certain missing components under certain circumstances, e.g., one or more information features 401 are missing. Accordingly, the input or initial patient data may be considered as “noisy” data. The denoising autoencoder 308 may be configured to denoise the initial patient data including missing data imputation, besides performing the feature extraction, and thus is ideally included in a parent symptom predictor 307.

To predict the probability of the cause of a reported symptom, the random forest-based classification may be further applied to the features extracted by the denoising autoencoder 308. Random forest classifier 310 is an inherent multi-class classifier consisting of a large number of relatively uncorrelated decision trees that operate as an ensemble, which can be used to classify an object based on features. Each individual tree in the random forest spits out a class prediction, where the class with the most votes becomes the model's prediction. For random forest classification, a sample of training set taken at random but with replacement is used to build a tree. When growing the tree, the best split is chosen among a random subset of the input features. As a result of this randomness, the model selects the classification/regression results that get the most votes from the trees in the forest, and thus help reduce the variance of the final model. Since a large number of relatively uncorrelated trees operating as a “committee” generally outperform any of the individual constituent models, a random forest classifier often demonstrates better performance than other classifiers. In addition, a random forest classifier generally is easy to tune and robust to overfitting, all of which makes the random forest classifier ideal to predict the parent symptom and/or complications for a reported symptom.

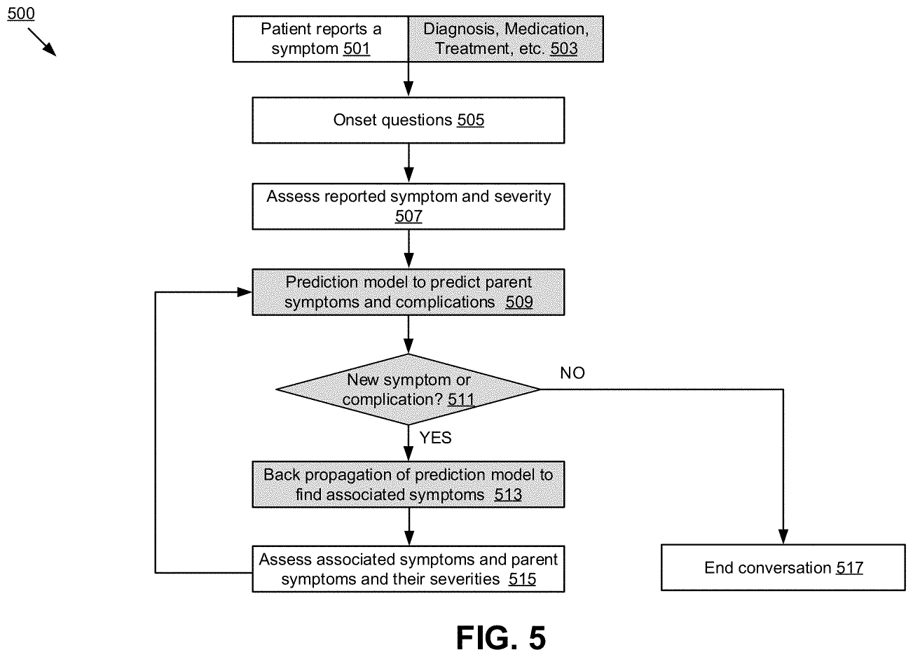

To train the prediction model (e.g., the parent symptom predictor 307) built on the denoising autoencoder 308 and the random forest classifier 310, data from a certain number of patients (e.g., 800 patients) along with bootstrapped data from clinical trials may be used for training. Each data may have certain features (e.g., 800 features) and a subset of features (e.g., 100 features) may be extracted by the denoising autoencoder 308, and the extracted features may be then fed into the random forest classifier 310 for training the classifier. During the training, to increase the robustness of the random forest classifier, five-fold cross-validation may be applied. The as-trained prediction model/parent symptom predictor 307 may allow an accurate prediction or identification of the parent symptom(s) and/or complication(s) 411 associated with a reported symptom, as further described below in FIG. 5.

FIG. 5 illustrates an example workflow 500 for prediction of parent symptoms and/or complications associated with a reported symptom, according to embodiments of the disclosure. As illustrated in FIG. 5, in step 501, a patient reports a symptom. Based on the reported symptom and/or the patient medical information 503 (such as diagnosis, medication, treatment, etc.), a set of onset questions are then presented to the patient in step 505. Next, in step 507, a set of assessment questions for the reported symptom are then presented to the patient for assessment of the reported symptom and the associated severity. The assessment of the reported symptom and the associated severity may be a dynamic process, which is implemented after each response is received from the patient during the symptom assessment.

In step 509, the parent symptom predictor 307 may predict parent symptom(s) and/or complication(s) associated with the reported symptom. The parent symptom predictor 307 may use the answers to the assessment questions as well as the medical information of the patient to predict the parent symptom(s) and/or complication(s) associated with the reported symptom. The parent symptom predictor 307 may use the trained denoising autoencoder 313 combined with the random forest classifier 315 to determine the potential parent symptom(s) and/or complication(s).

In step 511, the determined potential parent symptom(s) and complication(s) are then compared to the reported symptom. As previously described, a reported symptom may be a parent symptom. However, in many situations, either a new symptom or a complication is predicted. If there is no new symptom and/or complication predicted by the parent symptom predictor 307, the conversation ends in step 517. The digital parent application 107 may then assess and report the severity of the reported symptom (e.g., by using the medical severity classifier). In some embodiments, the potential risk may be also assessed and reported for the reported symptom.

If a new symptom and/or complication is predicted by the parent symptom predictor 307 in step 509, the parent symptom predictor 307 may be back propagated to find associated symptoms in step 513. Typically, a parent symptom or a complication causes multiple symptoms. Other than the predicted parent symptom, there are associated symptoms. To find the most likely associated symptoms, back propagation of the prediction model (or the parent symptom predictor 307) may be applied. All symptoms found through the back propagation will be sorted using a sequential forward feature selection (SFFS). The first symptom accounts for the largest variance and is the most likely contributor, and the second symptom accounts for the second largest variance and is the second likely contributor, and so on. A threshold may be applied to find the most likely symptom(s), which are then considered as the associated symptom(s). The threshold may be empirically selected, to make sure that not too many or too few symptoms are selected.

Next, in step 515, the patient is then prompted with questions to assess each predicted parent symptom, associated symptom, and/or complication if there is any. After the questions are answered, the first iteration of the symptom assessment is complete. All the information collected through the chatbot engine 105, combined with the previous information, may be then used for the next iteration of prediction by the prediction model (or parent symptom predictor 307) by returning the process to step 509. If a new associated symptom or complication is predicted, prediction back propagation will be used again to find associated symptoms. This time, the patient will only be assessed with the newly found symptoms by being prompted with questions related to the newly found symptoms that were not being asked in the previous iteration(s). In some embodiments, this process is iterated until no new symptoms and complications are predicted. In applications, the parameters for identifying potential symptoms/complications may be tuned to balance two competing factors: 1) to find as many potential parent/associated symptoms and/or complications as possible; and 2) to avoid asking patients too many questions, which may lead to drop off by the patient.

As previously described, when two patients report the same symptom, if patients' medical history and/or how patients answer each question is different, the set of assessment questions presented to the patients may be different. That is, different patients may have different pathways through the journey of digital nursing provided by the chatbot engine 105. In this aspect, a digital nursing system 100 may be also considered as a personalized digital nursing system.

Referring back to FIG. 3, in some embodiments, the digital nursing application 107 further includes a medical severity classifier 309 for determining the severity of each reported or identified symptom and complication.

The medical severity classifier 309 may be configured to determine the severity of the symptoms (e.g., reported symptom, parent symptom) and complications based on the associated parameters. The parameters provide an in-depth understanding of the symptoms and complications, and the severity describes how severe a symptom and complication is: non-urgent, urgent, or emergent. Based on the collected information for a given symptom, severity is determined. The logic for determining the severity is pre-defined according to certain standards (e.g., PRO CTCAE) and is built-in for each symptom. In one example, a scoring system may be applied to valuate patient responses to questions for a reported symptom. For each question answered by the patient, a score is assigned by the digital nursing system 100.

According to one embodiment, score assignment follows the following rules:

An answer gets a score of 1, if it's non-urgent quality.

An answer gets a score of 2, if it's urgent quality.

An answer gets a score of 3, if it's emergent quality.

An answer gets the highest score of multiple medical condition, if the answer includes multiple medical conditions.

The highest score is the final score. The final score may be then classified to a medical condition severity as shown in Table 1: chatbot conversation final score and corresponding symptom severity. In some embodiments, a clinic may further triage symptoms based on the final score.

TABLE 1

| Final Score | Medical Condition Severity |
|---|---|
| 1 | Non-urgent |
| 2 | Urgent |
| 3 | Emergent |

Here the following Table 2 provides one example chatbot conversation along with scores for a vomiting symptom.

TABLE 2

| Chatbot question, | | | | |
|---|---|---|---|---|
| options, and | | | | |
| assigned scores | Answer | Severity | Score | Notes |
| When did it start? | 1 day ago | Non- | 1 | Onset question, is |
| Within 24 hours | | urgent | | a contributing |
| 1 day ag | | | | factor of gastritis, |
| 2 days ago | | | | non-urgent |
| 3 days ago | | | | medical condition |
| In the last week | | | | |
| In the last month | | | | |
| How many times | 1-2 | Non- | 1 | PRO CTCAE |
| (separated by 5 | episodes | urgent | | measurement |
| minutes) have you | | | | |
| vomited in 24 | | | | |
| hours? | | | | |
| 1-2 episodes | | | | |
| 3-5 episodes | | | | |
| 6+ episodes | | | | |
| Did you vomit | Yes | Emergent | 3 | Emergent quality |
| bright red blood? | | | | |
| Yes | | | | |
| No | | | | |
| Have you been able | I am not | Emergent | 3 | Emergent quality |
| to drink any fluids | able to | | | |
| within this time | drink any | | | |
| period? | fluid in | | | |
| I drink 8+ glasses | the last | | | |
| of fluid per day | 24 hours | | | |
| I drink 3-8 glasses | | | | |
| of fluid per day | | | | |
| I drink 1-3 glasses | | | | |
| of fluid per day | | | | |
| I am not able to | | | | |
| drink any fluid in | | | | |
| the last 24 hours | | | | |
| Are you experi- | Upper | Non- | 1 | Is a contributing |
| encing any of | abdom- | urgent | | factor of Gastritis, |
| the following | inal | | | non-urgent |
| symptoms as well? | pain, | | | medical condition |
| Upper abdominal | Feeling | | | |
| pain | full | | | |
| Diarrhea | sooner | | | |
| Feeling full sooner | than | | | |
| than expected | expected | | | |
| Constipation | | | | |
| Final score | | Emergent | 3 | |

As can be seen from Table 2, the response to the question “When did it start?” is “1 day ago,” which is a contributing factor of gastritis, a non-urgent medical condition, which corresponds to score of 1. The response to the question “How many times (separated by 5 minutes) have you vomited in 24 hours?” is “1-2 episodes,” which gets a score of 1, since the answer has a non-urgent quality. The response to the question “Did you vomit bright red blood?” is “Yes,” which gets a score of 3, since the question has an emergent quality. The response to the question “Have you been able to drink any fluids within this time period?” is “I am not able to drink any fluid in the last 24 hours,” which gets a score of 3, since the question has an emergent quality. The response to the question “Are you experiencing any of the following symptoms as well?” is “Upper abdominal pain, felling full Sonner than expected.” Both are contributing factors of gastritis, a non-urgent medical condition, which leads to a score of 1. The highest score is 3, and therefore the final score for the medical condition is 3. According to the criteria defined in Table 1, the score of 3 means that the medical condition related to the reported symptom is then determined to be emergent. In some embodiments, the severity for other associated symptoms, parent symptoms, and/or complications may be similarly determined.

It is to be noted that, in real applications, for a patient's medical condition that includes the reported symptom, associated symptoms, parent symptoms, and potential complications, the severity of the most severe symptom may be defined as the severity of the medical condition. For instance, if at least one symptom is emergent, the medical condition will be emergent. Only if all symptoms are non-urgent, the medical condition will be non-urgent.

Referring back to FIG. 3, in some embodiments, the disclosed digital nursing application 107 may further include a risk assessment module 311 configured to assess the risk for the reported and identified symptoms and/or complications. The risk assessment module 311 may assess the potential risk of a patient based on the patient's responses to the assessment questions as well as the medical information of the patient in view of future developments and/or progresses. For instance, while the medical severity classifier 309 determines that the patient's symptom is not emergent at the moment of reporting nausea, the risk assessment module 311 may predict that the patient is at a high risk of intestinal obstruction, and thus still recommend an immediate care by a healthcare provider. In some embodiments, the risk assessment module 311 may assess the potential risk for the patient according to certain triage pathways. Additionally or alternatively, certain machine learning models may be used to predict the future disease state of a patient based on the patient's current medical condition and available history, and thus may be included in the risk assessment module 311.

In some embodiments, after the severities for the symptoms, complications, and medical condition and the potential risk are determined, a summary of the medical condition of the patient may be automatically generated and reported to the patient.

In some embodiments, depending on the determined severity, certain additional actions may be necessary for the benefit of the patient, after the determination of the symptom severity. Accordingly, the digital nursing application 107 may further include a post assessment action module 311 configured to determine appropriate actions to be taken based on the determined severity and the assessed risk. For instance, the assessment action module 311 may determine whether a notice should be generated and a healthcare provider should be notified for the identified severity and potential risk, whether an emergency alert should be generated to require an emergency dispatch, whether and/or when a follow-up symptom check should be scheduled, etc.

In conditions if a follow-up symptom check should be scheduled, the post assessment action module 311 may automatically schedule a follow-up check to check the progress of the reported symptom. Here the following are certain example follow-up questions that may be presented to the patient through a chatbot:

Value
Does symptom management reach the goal of the patient?

Provoking/Palliating
Does the patient feel better?

Impact
How does it impact a patient's ADL?

Region/radiation
Where does the symptom occur?
What is the progressing pattern?

Grade
Grade the symptom. Depending on the symptoms, different standards were followed, and they are presented in patient language.

Associated symptoms
What other symptoms occur at the same time?

Intervention
What is the intervention? e.g., adopted clinical advice, tried other methods or medication

Additional notes
A patient can leave additional notes.
   The responses for the follow-up questions may be further assessed for the severity and/or potential risk, similar to the assessment for a reported symptom as previously described.

Under certain circumstances (e.g., when a symptom is moderate severe but not emergent), the post assessment action may require a notice to be sent to the healthcare providers to seek advice. Accordingly, the post assessment action module 313 may optionally include a healthcare provider communication module 314 configured to transmit the reported symptom and the further determined severity and assessed risk to healthcare providers (e.g., nurse or physician), or upload this information to a user account associated with the patient so that the healthcare providers may check the transmitted or uploaded information for the patient. After a review of the reported information, a healthcare provider may provide instruction to the patient. The instruction may be feedbacked (e.g., through the same healthcare provider communication module 314) to the patient as a notification of a cloud-based app, as a text message, as a chat log posted at the end of the chat communication for reporting the symptom, etc. The patient can then follow the instructions provided by the healthcare provider without requiring to see the healthcare providers.

Under certain circumstances, an emergency alert may be necessary if the symptom/medical condition is determined to be emergent. The post assessment action module 313 may thus further include an emergency alert transmission module 316 configured to establish an emergency communication session between a patient and an appropriate emergency service provider. The emergency service provider may be a local emergency dispatch center, a healthcare provider, or another entity that can offer instant assistance to patients. In some embodiments, depending on the severity of the identified medical condition, the emergency alert transmission module 316 may automatically identify an appropriate entity for transmitting an alert. For instance, if the symptom is determined to be life-threatening, the emergency alert transmission module 316 may automatically dial a number corresponding to a local emergency dispatch center, so that the request for immediate action can be timely delivered to the emergency dispatch center for the benefit of the patient.

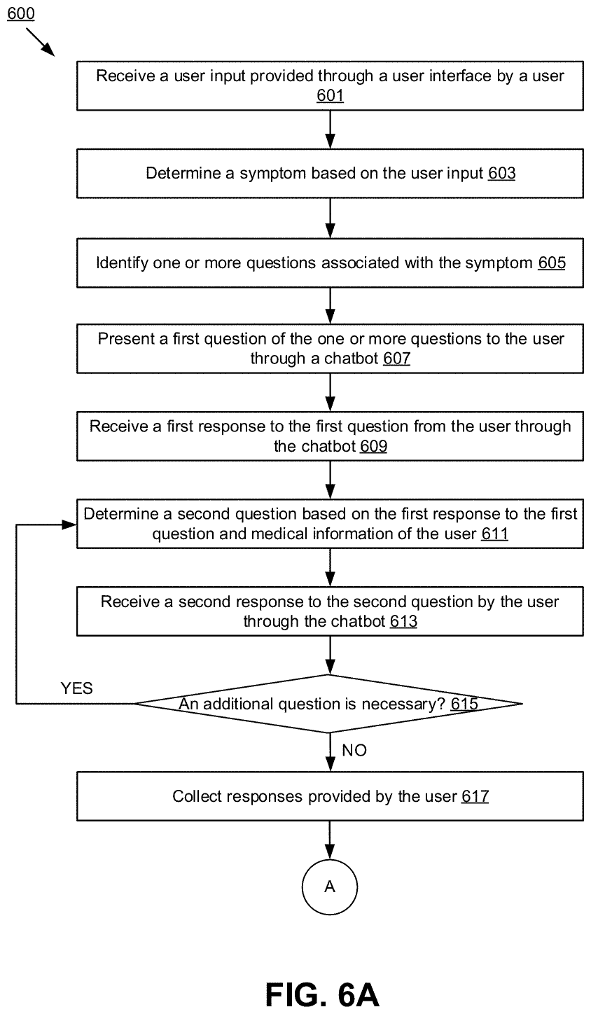

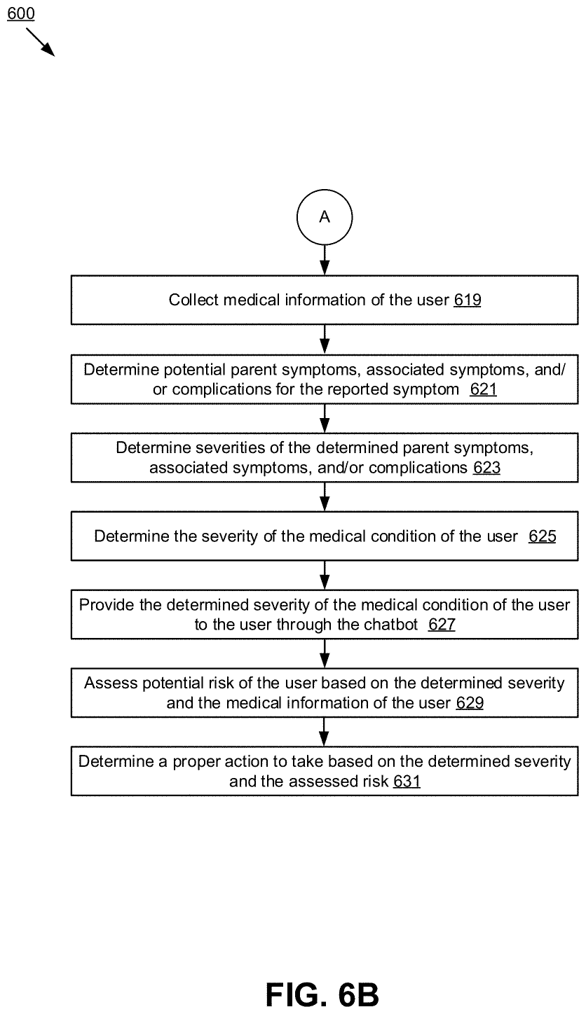

The above-described components or modules in FIG. 3 are provided for illustrative purposes. In some embodiments, the digital nursing application 107 may include additional or fewer components than those illustrated in FIG. 3. For instance, in some embodiments, the digital nursing application 107 may further include a healthcare provider management module (not shown) that allows one or more healthcare providers to access the patients' profile, manage symptoms, follow trends, and perform analysis, and communicate with the patients. The specific functions of the digital nursing application 107 are further described in detail with reference to the drawings in FIGS. 6A-8C.

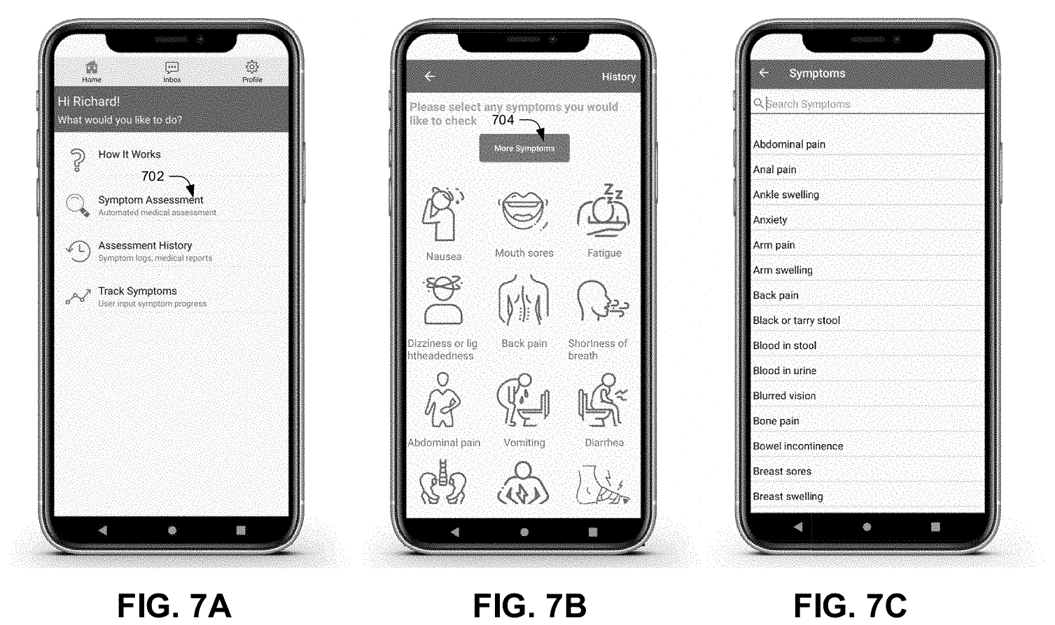

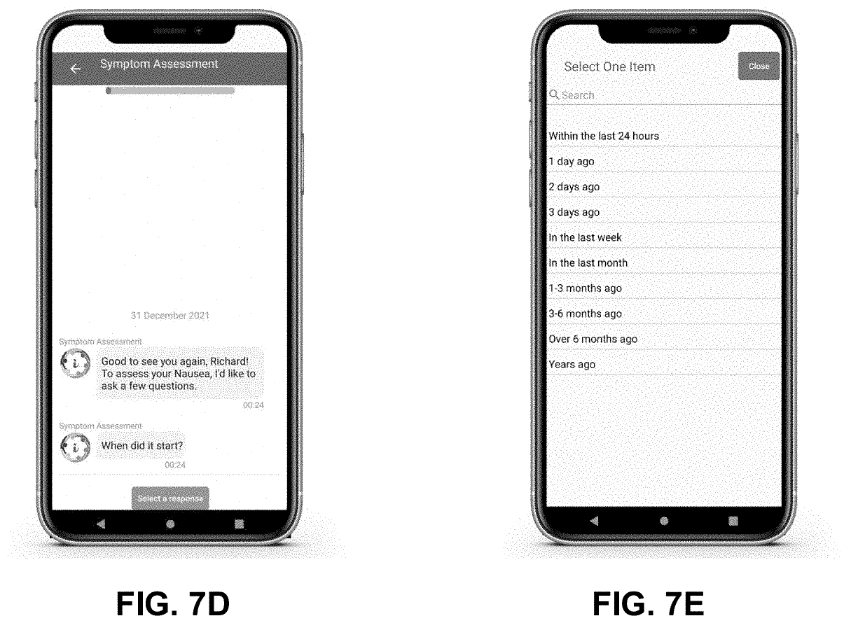

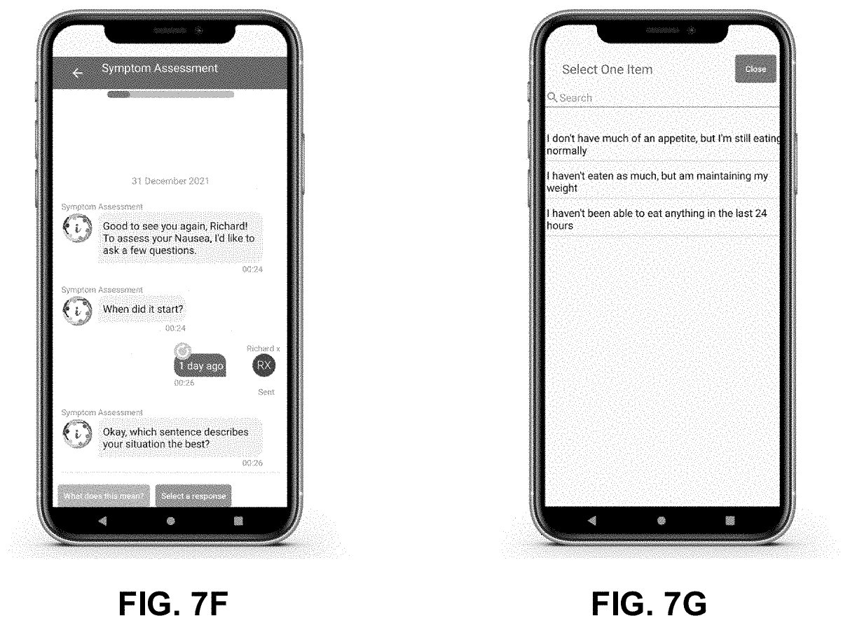

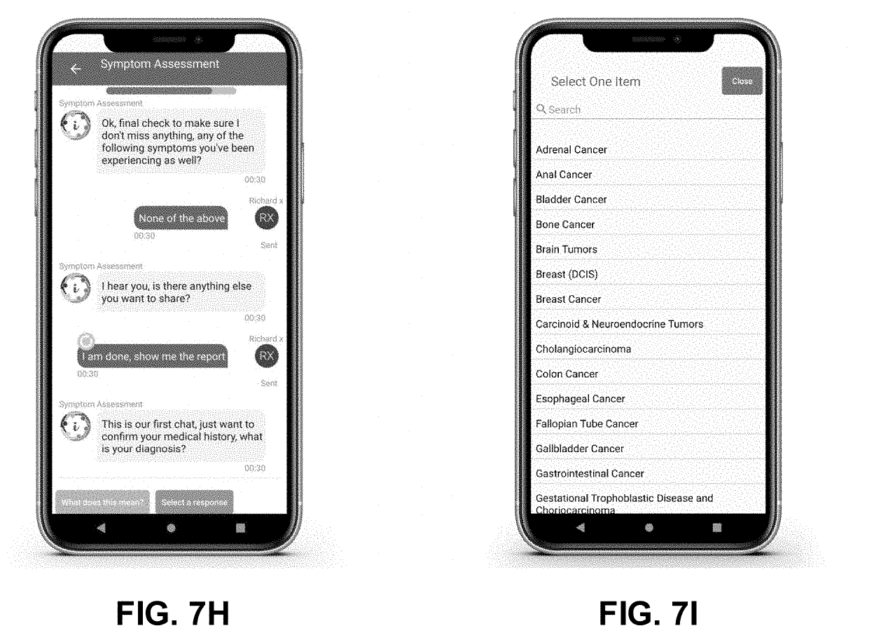

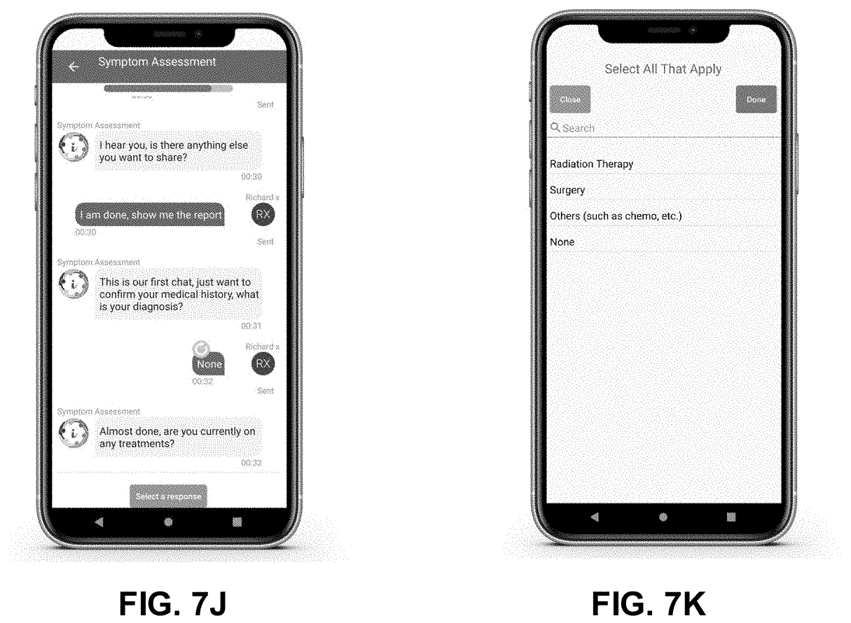

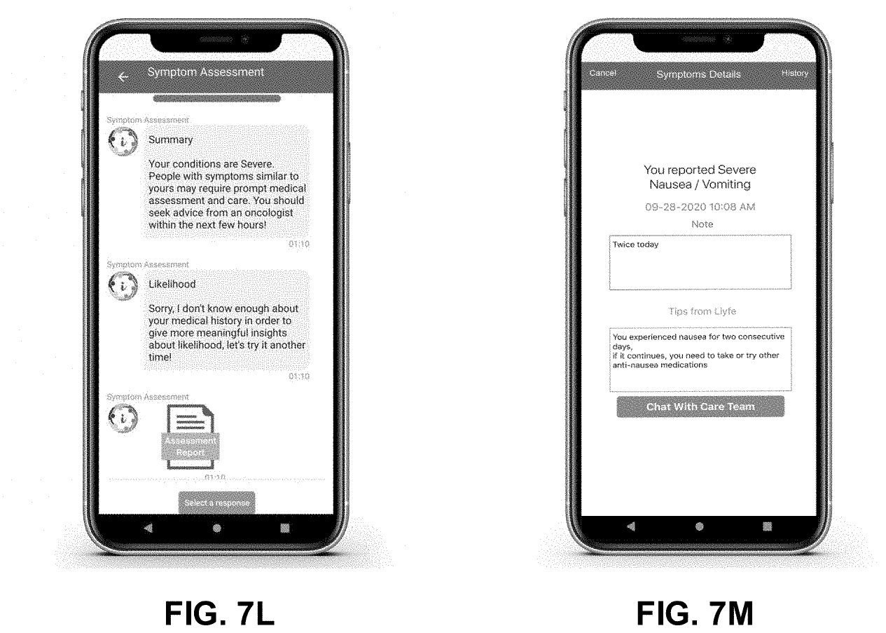

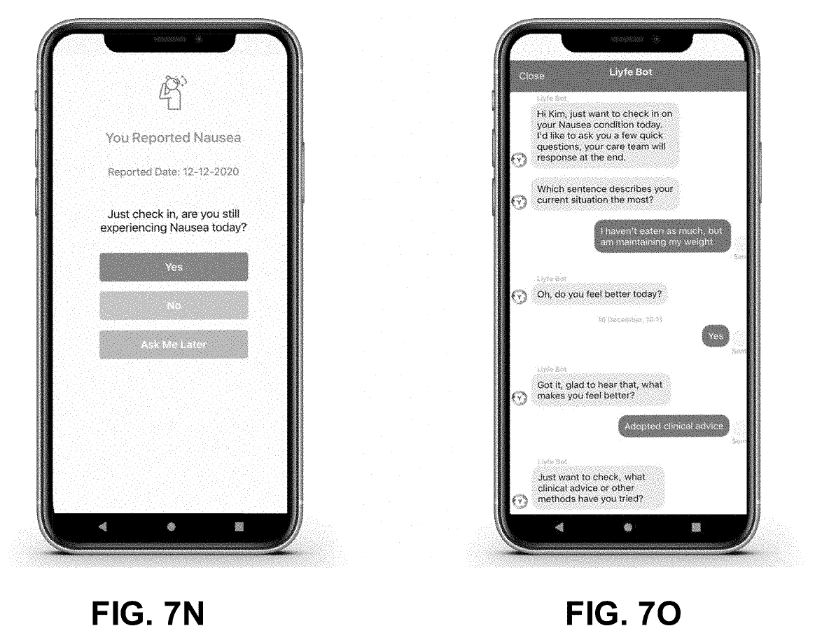

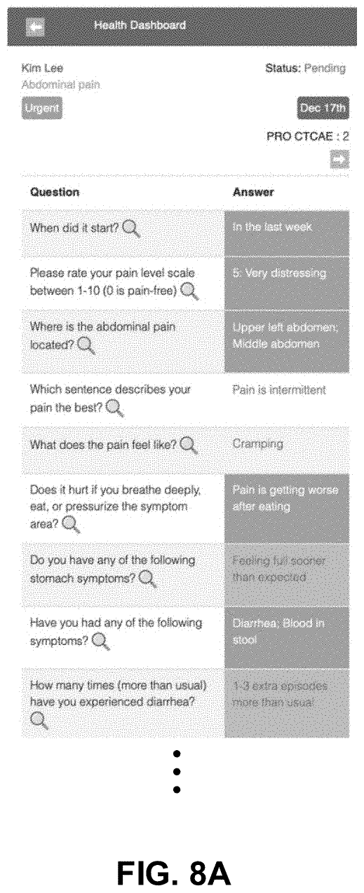

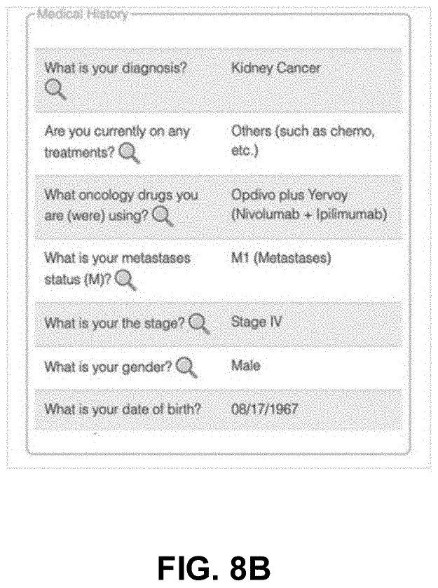

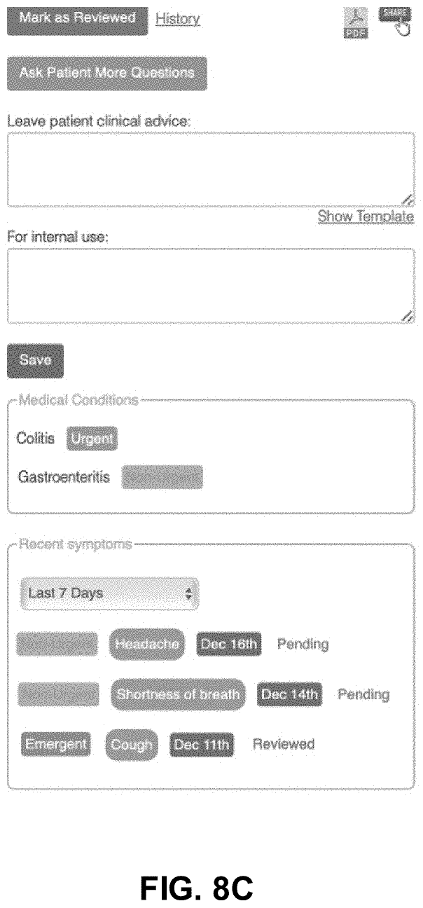

FIGS. 6A-6B collaboratively illustrate an example method 600 for assessing a symptom reported by a patient, FIGS. 7A-7O illustrate example patient-side user interfaces under different scenarios and FIGS. 8A-8C illustrate example healthcare provider-side user interfaces, according to embodiments of the disclosure. The specific processes in method 600 will be described in detail below in view of the user interfaces illustrated in FIGS. 7A-8C.

Method 600 starts with the receipt of user input through a user interface by a user in step 601. The user may be a patient, and the user interface may be a user interface of a digital nursing system 100 for symptom assessment, as shown in FIGS. 7A-7C. For instance, the user may click the “Symptom Assessment” 702 in FIG. 7A to start a symptom assessment. Once clicked, another user interface may pop up, showing the most frequently reported symptoms as symbols, as shown in FIG. 7B. The user may select a symbol from the user interface to start to report a symptom the user hopes to. If the user cannot find a symptom from the displayed symbols, the user may click “More Symptoms” 704, so that a long list of symptoms may be displayed in another user interface, as shown in FIG. 7C. Once a symptom is selected by the user, the digital nursing system may determine a symptom to be reported based on the user input from the user in step 603. Here, the user input may specifically refer to a user input for the user to select a target symptom s/he wants to report. In some embodiments, if the symptom is not displayed as a symbol or included in the list, the user may be allowed to input a text, voice, or image, as previously described to report a symptom. The digital nursing system 100 may identify the symptom to be reported based on the information interpreted from the text, voice, or image input (e.g., by the natural language processor 301 included in the digital nursing system 100).

In step 605, the digital nursing system 100 may identify one or more questions associated with the to-be-reported symptom, and present the associated questions to the user in a chatbot configured for symptom assessment in step 607, as further described in detail below. FIG. 7D illustrates a user interface for starting the symptom assessment. As can be seen, the chat communication may start with a greeting message from the digital nursing system. In the next, a first question “When did it start?” may be then presented to the user. The first question may be an onset question that discovers the date when the user initially experienced the reported symptom. As illustrated in FIG. 7D, when the first question is presented, the user may be also prompted with an option to select a response from a predefined list of responses prepared by the digital nursing system 100, as shown in FIG. 7E. In some embodiments, the user may also have an option to customize his/her answer with a text or voice input, which can be also recognized by the digital nursing system 100.

In step 609, the digital nursing system 100 receives a first response to the first question from the user through the chatbot. For instance, the user may select a response from the list shown in FIG. 7E, or provide a text or voice input through the chatbot in response to the first onset question. Once the response is selected or input, the selected or identified response (e.g., based on the text or voice input) may be then presented in the chatbot as a reply message, as “1 day ago” shown in FIG. 7F. As also shown in FIG. 7F, the reply message “1 day ago” may be recalled in case the user made a wrong selection or changed his/her mind.

In step 611, the digital nursing system 100 may determine a second question based on the first response to the first question and the medical information of the user. The medical information of the user may be retrieved from the user account that stores medical information including the previous medical history of the user. Under certain circumstances, the medical information of the user may be also retrieved from a third-party service provider, such as a medical institute, a health department, or a hospital that is ready to share patient information upon request and upon privacy agreement provided by the user. In some embodiments, if no medical information of the user is currently available, the user may be prompted to provide such medical information through the chatbot, as described in detail later.

In some embodiments, the digital nursing system 100 may apply a machine learning model to identify a proper second question to ask. The machine learning model may be trained based on the patient medical history of a lot of patients so that the most relevant question is to be asked for an assessment of the reported symptom. The trained machine learning model may be fed with the medical information of the user as well as the first response to the first question. For instance, the digital nursing system 100 may determine that the second question is “Okay, which sentence describes your situation the best?” as illustrated in FIG. 7F. Here, the term “Okay” may be added due to the conversation tone randomization as previously described. As shown in FIG. 7F, when presenting the second question to the user in the chatbot, besides the response options for selection by the patient, the digital nursing system 100 may also provide a link for explaining the question in case that the patient is not sure what the system 100 is asking for. This may help avoid misunderstanding during the symptom assessment. FIG. 7G further provides a list of selectable responses from which the patient can select. The responses are provided for example purposes. It should be noted that for different patients, different response lists may be provided so that the most relevant information for symptom assessment for that specific person can be collected. In step 613, the digital nursing system may receive a second response to the second question by the user through the chatbot.

In step 615, the digital nursing system 100 may determine whether an additional question is necessary for the symptom assessment. In some embodiments, the digital nursing system 100 may simply check whether there is any remaining question in the identified one or more associated questions in step 605. If there is any, the digital nursing question can return to step 611 to determine a remaining question to ask, until there is no remaining question in the identified one or more questions identified for the reported symptom. Under certain circumstances, however, even when there are still one or more questions remaining, if the currently available responses to the already asked questions indicate that the medical condition of the patient is clearly emergent, the digital nursing system 100 may immediately make a decision that the medical condition of the patient is emergent. At this stage, the digital nursing system 100 may terminate the symptom assessment, to save time for the patient so that proper action can be timely taken for the benefit of the patient.

In step 617, after responses to all questions are completed, the digital nursing system 100 may collect the responses to the questions. The collected responses, along with the medical information of the patient, may be further used for predicting potential parent symptoms, associated symptoms, and/or complications and assessment of their corresponding severities, as further described more in detail later in FIG. 6B.

In step 619, the digital nursing system 100 may collect the medical information of the user. In some embodiments, the medical information of the user may be already collected, as described above in step 611. Under certain circumstances, the medical information of the user may be not readily available, for example, when the user is a new customer of the digital nursing system 100. At this point, the digital nursing system 100 may collect the medical information of the user through a chatbot. For example, the digital nursing system 100 may ask the user's medical history, such as the diagnosis shown in FIG. 7H and treatments shown in FIG. 7J. The user may then provide the medical history and treatment information by responding to these questions, as shown in FIGS. 71 and 7M. It should be noted that questions for asking medical information of the user are for exemplary purposes only. In applications, the digital nursing system 100 may ask for any medical information that the system considers as necessary to make a decision in the symptom assessment.

In step 621, the digital nursing system 100 may determine potential parent symptoms, associated symptoms, and/or complications for the reported symptom. The digital nursing system 100 may apply the denoising autoencoder 313 and random forest classifier 315 to identify these symptoms and complications, as described earlier in FIGS. 3-4.

In step 623, the digital nursing system 100 may determine the severities of the identified parent symptoms, associated symptoms and/or complications, and in step 625 further determine the severity of the medical condition of the user based on the determined severities for the reported symptom, determined parent symptoms, associated symptoms, and/or complications. In some embodiments, the medical condition of the user is determined based on the severity of the most severe symptom/complication, as described earlier.

In step 627, the digital nursing system 100 may present the determined severity to the user through the chatbot, as shown in the “Summary” section in FIG. 7L. In some embodiments, a downloadable version of the assessment report may be also delivered to the user through the chatbot, as also shown by “Assessment Report” in FIG. 7L. In some embodiments, possible causes of the reported symptom may be also delivered to the user, as shown in the “Likelihood” section in FIG. 7L.

In step 629, the digital nursing system 100 may assess the potential risk of the user based on the determined severity and the medical information of the user in view of future development of the disease(s) associated with the symptom(s), as described earlier.

In step 631, the digital nursing system 100 may determine a proper action to take based on the determined severity of the medical condition of the user and the assessed risk. The possible actions may include sending a notice to an associated healthcare provider, contacting a local emergency dispatch center, scheduling a follow-up symptom assessment, as described earlier.

FIGS. 7N-7O illustrate example user interfaces for a follow-up symptom assessment. As can be seen from the figures, the digital nursing system 100 may ask whether the reported symptom still remains after a certain period of time. Additionally or alternatively, the digital nursing system 100 may ask the questions for the current condition of the user as shown in FIG. 7O, in a way similar to the assessment of the previously reported symptom. By enabling timely and automatic follow-ups, it can be ensured that the patients are not suffering from the same symptoms for a long time due to intentional or unintentional neglect.

In some embodiments, besides the user interfaces configured to be accessible by the patients as shown in FIGS. 7A-7O, the digital nursing system 100 may also include certain user interfaces configured for assessment by the healthcare providers so that the medical conditions of the patients can be timely monitored. FIGS. 8A-8C illustrate example user interfaces accessible to the healthcare providers. These user interfaces may be web-version user interfaces, different from the mobile phone version of user interfaces shown in FIGS. 7A-7O.

Specifically, FIG. 8A displays a section or a user interface including the details of the assessed symptom recently reported by a patient, which includes the questions asked and responses provided by the patient during the symptom assessment. The determined medical condition of the patient is also displayed. FIG. 8B displays another section or user interface including the medical information or medical history of the patient, which includes the diagnosis, the treatments, the medication, the disease status, user profile information, as illustrated in the figure.

FIG. 8C displays yet another section providing a user interface to allow the healthcare providers to leave notes, provide instruction, track the symptoms, and so on. The instructions and/or notes directed to the patients, once input by the healthcare providers, may be delivered to a corresponding patient, e.g., as a notification transmitted to a client device of the patient, which when clicked, may allow the instruction to be presented to the patient. FIG. 7M illustrates an example user interface for displaying a piece of instruction transmitted and presented to a patient, which directs the patient to take or try other anti-nausea medications if the symptom (e.g., nausea) continues. In this way, the patient may get well care from the healthcare provider without requiring the patient to actually visit the healthcare provider.

Although the techniques have been described in language specific to structural features and/or methodological acts, it is to be understood that the subject matter defined in the appended claims is not necessarily limited to the specific features or acts described. Rather, the specific features and acts are disclosed as example forms of implementing the claimed subject matter, and other equivalent features and methods are intended to be within the scope of the appended claims. Further, various different embodiments are described and it is to be appreciated that each described embodiment can be implemented independently or in connection with one or more other described embodiments.

What is claimed is:

1. A system for intelligent symptom assessment through a machine learning-driven digital assistance platform, the system comprising:
a processor; and
a memory, coupled to the processor, configured to store executable instructions that, when executed by the processor, cause the processor to:
process user input received from a user device communicating with a chatbot environment, the user input indicating a user request for assessing a symptom in the chatbot environment;
generate one or more questions related to the symptom and communicate the one or more questions to the user device in the chatbot environment;
receive, in the chatbot environment, responses to the one or more questions provided by the user;
collect additional medical information associated with the user; and
determine one or more parent symptoms or complications associated with the symptom based on the one or more responses provided by the user and the additional medical information of the user.

2. The system of claim 1, wherein the executable instructions further include instructions that, when executed by the processor, cause the processor to:
determine severity of each of the symptom, the one or more parent symptoms, or the one or more complications; and
determine a medical condition of the user based on the determined severity of each of the symptom, the one or more parent symptoms, or the one or more complications.

3. The system of claim 2, wherein the executable instructions further include instructions that, when executed by the processor, cause the processor to:
determine a proper action to take based on the determined medical condition of the user.

4. The system of claim 3, wherein, the executable instructions further include instructions that, when executed by the processor, cause the processor to:
in response to the determined medical condition of the user is not emergent, transmit a notice to a healthcare provider.

5. The system of claim 3, wherein, the executable instructions further include instructions that, when executed by the processor, cause the processor to:
in response to the determined medical condition of the user is not emergent, automatically schedule a follow-up symptom assessment to check the user in the chatbot environment at a later time.

6. The system of claim 3, wherein, the executable instructions further include instructions that, when executed by the processor, cause the processor to:
in response to the determined medical condition of the user is emergent, automatically contact an emergency dispatch center to seek timely assistance for the user.

7. The system of claim 1, wherein a first question of the one or more questions is generated according to a predefined rule.

8. The system of claim 7, wherein each of the remaining of the one or more questions is generated according to responses of the user to one or more preceding questions.

9. The system of claim 7, wherein each of the remaining of the one or more questions are generated according to the additional medical information of the user along with responses of the user to one or more preceding questions.

10. The system of claim 1, wherein the one or more parent symptoms or complications associated with the symptom are determined by a prediction model constructed based on a denoising autoencoder combined with a random forest classifier.

11. The system of claim 10, wherein the denoising autoencoder is a three-layer denoising autoencoder.

12. The system of claim 10, wherein the executable instructions further include instructions that, when executed by the processor, cause the processor to:
determine whether a new symptom is identified based on the determined one or more parent symptoms or complications; and
response to a new symptom being identified, determining one or more associated symptoms for the identified new symptom.

13. The system of claim 12, wherein the one or more associated symptoms is identified by back propagation of the prediction model.

14. The system of claim 12, wherein the executable instructions further include instructions that, when executed by the processor, cause the processor to:
determine severity of each of the one or more associated symptoms.

15. A method for intelligent symptom assessment through a machine learning-driven digital assistance platform, the method comprising:
processing a user input received from a user device communicating with a chatbot environment, the user input indicating a user request for assessing a symptom in the chatbot environment;
generating one or more questions related to the symptom and communicating the one or more questions to the user device in the chatbot environment;
receiving, in the chatbot environment, responses to the one or more questions provided by the user;
collecting additional medical information associated with the user; and
determining one or more parent symptoms or complications associated with the symptom based on the one or more responses provided by the user and the additional medical information of the user.

16. The method of claim 15, further comprising:
determining severity of each of the symptom, the one or more parent symptoms, or the one or more complications; and
determining a medical condition of the user based on the determined severity of each of the symptom, the one or more parent symptoms, or the one or more complications.

17. The method of claim 16, further comprising:
determining a proper action to take based on the determined medical condition of the user.

18. The method of claim 15, wherein the one or more parent symptoms or complications associated with the symptom are determined by a prediction model constructed based on a denoising autoencoder combined with a random forest classifier.

19. The method of claim 15, further comprising:
determining whether a new symptom is identified based on the determined one or more parent symptoms or complications; and
response to a new symptom being identified, determining one or more associated symptoms for the identified new symptom.

20. A computer program product for inputting text on-site in a drawing or art software application, the computer program product comprising a non-transitory computer-readable medium having computer-readable program code stored thereon, the computer-readable program code configured to:
process user input received from a user device communicating with a chatbot environment, the user input indicating a user request for assessing a symptom in the chatbot environment;
generate one or more questions related to the symptom and communicate the one or more questions to the user device in the chatbot environment;
receive, in the chatbot environment, responses to the one or more questions provided by the user;
collect additional medical information associated with the user; and
determine one or more parent symptoms or complications associated with the symptom based on the one or more responses provided by the user and the additional medical information of the user.
